(19) United States
(12) Patent Application Publication
(10) Pub. No.: US 2017/0265844 A1
(43) Pub. Date: Sep. 21, 2017

(54) ULTRASONIC PROBE AND ULTRASONIC DEVICE

(71) Applicant: Seiko Epson Corporation, Tokyo (JP)

(72) Inventor: Tsutomu NISHIWAKI, Azumino (JP)

(21) Appl. No.: 15/447,566

(22) Filed: Mar. 2, 2017

(30) Foreign Application Priority Data
Mar. 18, 2016 (JP) 2016-054950

(51) Int. Cl. A61B 8/00; A61B 1/04

(52) U.S. Cl. CPC A61B 8/4472; A61B 1/041; A61B 8/54

(57) ABSTRACT

An ultrasonic probe includes ultrasonic vibrators arranged in a circumferential direction, an acoustic matching layer disposed on the ultrasonic vibrators, and a flexion section disposed between the ultrasonic vibrators adjacent to each other in the circumferential direction, and having an electrode layer acting as an electrode of the ultrasonic vibrators adjacent to each other. Further, it is also possible to configure the ultrasonic probe in which the electrode layer is exposed in the flexion section.

### BACKGROUND

1. Technical Field

The present invention relates to an ultrasonic probe for transmitting and receiving an ultrasonic wave.

2. Related Art

In the past, there has been known a small-sized ultrasonic probe introduced and then used in the body of a test subject. For example, in JP-A-2007-181592 (Document 1), there is disclosed an ultrasonic capsule endoscope having a plurality of probes (ultrasonic vibrators) for transmitting and receiving an ultrasonic wave arranged along the internal peripheral surface of the main body.

Incidentally, in the case of incorporating a plurality of ultrasonic vibrators in such a capsule-type small-sized ultrasonic probe as in the technology of Document 1, the arrangement space is limited, and therefore, high-density packaging of the ultrasonic vibrators is required. However, Document 1 fails to describe a specific packaging method of the plurality of ultrasonic vibrators although including the description of the arrangement of the plurality of ultrasonic vibrators.

### SUMMARY

An advantage of some aspects of the invention is to realize a small-sized ultrasonic probe having a plurality of ultrasonic vibrators densely packaged.

A first aspect of the invention is directed to an ultrasonic probe including a plurality of ultrasonic vibrators arranged in a circumferential direction, an acoustic matching layer disposed on the ultrasonic vibrators, and a flexion section disposed between the ultrasonic vibrators adjacent to each other in the circumferential direction, and having an electrode layer acting as an electrode of the ultrasonic vibrators adjacent to each other.

According to the first aspect of the invention, by disposing the electrode layer acting as the electrode of the ultrasonic vibrators between the ultrasonic vibrators adjacent to each other in the circumferential direction to bend the electrode layer, the arrangement of the ultrasonic vibrators in the circumferential direction can be realized. Since the electrode layer acts as the electrode between the ultrasonic vibrators adjacent to each other, the arrangement intervals of the ultrasonic vibrators can be narrowed, and further, both of the retainment of the binding force for connecting the ultrasonic vibrators adjacent to each other, and the flexibility can be achieved by the electrode layer. Therefore, it is possible to densely package the plurality of ultrasonic vibrators, and further miniaturization of the ultrasonic probe can be achieved.

A second aspect of the invention is directed to the ultrasonic probe according to the first aspect of the invention, in which the electrode layer is exposed in the flexion section.

According to the second aspect of the invention, it is possible to expose the electrode layer between the ultrasonic vibrators adjacent to each other in the circumferential direction.

A third aspect of the invention is directed to the ultrasonic probe according to the first or the second aspect of the invention, in which the acoustic matching layer is disposed on the ultrasonic vibrators and between the ultrasonic vibrators, and formed of a material having flexibility, and a principal element in a thickness direction of the flexion section is constituted by the acoustic matching layer and the electrode layer.

A fourth aspect of the invention is directed to the ultrasonic probe according to any one of the first through third aspects of the invention, in which the flexion section has the electrode layer on one end side in a thickness direction, and has a groove section adapted to improve flexibility on an end surface on the other end side.

According to the third aspect of the invention, the flexibility of the flexion section can be improved. Further, according to the fourth aspect of the invention, the flexibility can further be improved.

A fifth aspect of the invention is directed to the ultrasonic probe according to any one of the first through fourth aspects of the invention, in which the ultrasonic vibrators are circularly arranged with an ultrasonic radiation direction facing outward, and an ultrasonic wave can be transmitted and received throughout an entire circumference.

A sixth aspect of the invention is directed to the ultrasonic probe according to the fifth aspect of the invention, in which N (N≧2) element parts each including the ultrasonic vibrators, the acoustic matching layer, and the flexion part are arranged in the circumferential direction to configure the circular arrangement of the ultrasonic vibrators.

According to the fifth aspect of the invention, it is possible to transmit and receive the ultrasonic wave in the directions of the entire circumference of the plurality of ultrasonic vibrators arranged circularly from the plurality of ultrasonic vibrators arranged circularly. Further, according to the sixth aspect of the invention, the circular arrangement of the ultrasonic vibrators can be realized by the arrangements of the element parts each having the ultrasonic vibrators, the acoustic matching layer, and the flexion section.

A seventh aspect of the invention is directed to the ultrasonic probe according to anyone of the first through sixth aspects of the invention, which further includes a shell part incorporating all of the constituents to introduce the ultrasonic probe in a body cavity.

According to the seventh aspect of the invention, it is possible to realize the ultrasonic probe which can be introduced in the body cavity.

An eighth aspect of the invention is directed to the ultrasonic probe according to the seventh aspect of the invention, in which the shell part has an elongated shape, the ultrasonic vibrators are provided to a body part of the shell part, and an ultrasonic wave is radially emitted viewed from a longitudinal direction of the shell part.

According to the eighth aspect of the invention, it is possible to radially emit the ultrasonic wave viewed from the longitudinal direction of the shell part in the body cavity.

A ninth aspect of the invention is directed to the ultrasonic probe according to one of the seventh and eighth aspects of the invention, in which the shell part has a capsule shape.

According to the ninth aspect of the invention, it is possible to realize the capsule-type ultrasonic probe.

### BRIEF DESCRIPTION OF THE DRAWINGS

The invention will be described with reference to the accompanying drawings, wherein like numbers reference like elements.

FIG. 1 is a schematic diagram showing an internal configuration example of an ultrasonic probe according to an embodiment of the invention.

FIG. 2 is a diagram schematically showing a cross-sectional surface viewed along the arrowed line A-A shown in FIG. 1.

FIG. 3 is a cross-sectional view showing a manufacturing process of a vibrator plate.

FIG. 4 is a cross-sectional view showing another manufacturing process of the vibrator plate.

FIG. 5 is a cross-sectional view showing another manufacturing process of the vibrator plate.

FIG. 6 is a cross-sectional view showing another manufacturing process of the vibrator plate.

FIG. 7 is a perspective view showing an assembly procedure.

FIG. 8 is another perspective view showing the assembly procedure.

FIG. 9 is another perspective view showing the assembly procedure.

FIG. 10 is another perspective view showing the assembly procedure.

FIG. 11 is a cross-sectional view of an ultrasonic probe according to a modified example.

FIG. 12 is a perspective view showing a fixation member in the modified example.

FIG. 13 is a schematic diagram showing an internal configuration example of an ultrasonic probe according to another modified example.

FIG. 14 is a perspective view schematically showing a vibrator plate.

FIG. 15 is a plan view showing a developed state of the vibrator plate.

### DESCRIPTION OF AN EXEMPLARY EMBODIMENT

Hereinafter, a preferred embodiment of the invention will be described with reference to the accompanying drawings. In the following description, there is illustrated a capsule-type ultrasonic probe used in a situation of being swallowed by a human. It should be noted that the invention is not limited by the embodiment hereinafter described, and configurations to which the invention can be applied are not limited to the following embodiment. Further, in the description of the drawings, the same parts are denoted by the same symbols.

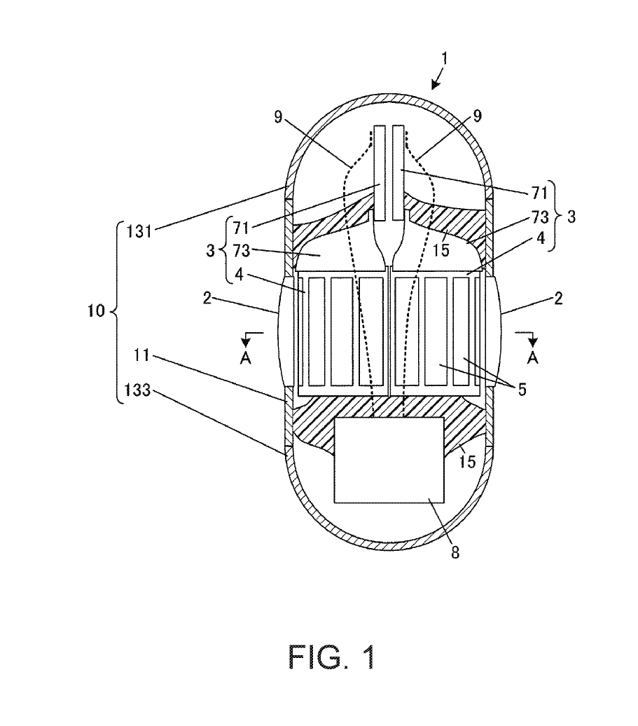

FIG. 1 is a schematic diagram showing an internal configuration example of the ultrasonic probe 1 according to the present embodiment, and shows an internal condition by cutting out a side surface of a capsule-type housing 10 as a shell part. The ultrasonic probe 1 is introduced in the body cavity of a test subject, and transmits and receives an ultrasonic wave to perform ultrasonic measurement in the body cavity. A received signal of the reflected wave thus obtained is wirelessly transmitted as needed basis to a receiver (not shown) located outside the body as a measurement result. The measurement result received by the receiver is imaged as needed basis by, for example, an image generation device connected to achieve communication with the receiver, and is then used for diagnosis or the like. Further, it is also possible to adopt a configuration of storing the received signal in a time-series manner in an IC (integrated circuit) memory incorporated, and then retrieve the received signal from the ultrasonic probe 1 discharged outside the body to be used for making a picture.

The capsule-type housing 10 is provided with a size in a range enough for a human to swallow, and houses (incorporates) to protect internal constituents (embedded components) of the ultrasonic probe 1. For example, the capsule-type housing 10 is constituted by a cylindrical member 11 having a cylindrical shape for forming a body part of the capsule-type housing 10, and cover members 131, 133 each having a semispherical shape to be fitted respectively into both ends of the cylindrical member 11 to close the openings. When housing the internal constituents, the internal gap is filled with a resin material 15 or the like to thereby fix each of the internal constituents, and the fitting sections between the cylindrical member 11 and the cover members 131, 133 are bonded, and thus the inside is sealed. It should be noted that it is sufficient for the capsule-type housing 10 to have an elongated shape having the body part, and the outer shape of the capsule-type housing 10 is not limited to the cylindrical shape having the semispherical shapes at the both ends shown in the drawings and also called an “extended elliptical shape,” but can also be a capsule shape such as a “football shape” having an elliptical cross-sectional shape. Further, although a three-piece structure having the cylindrical member 11 and the upper and lower cover members 131, 133 is adopted as the configuration of the capsule-type housing 10, it is also possible to adopt a two-piece structure in which, for example, either one of the cover members 131, 133 has an integrated structure with the cylindrical member 11.

In a sidewall of the cylindrical member 11, there are fitted the same number of acoustic lenses 2 as the number of ultrasonic vibrators arranged in a circumferential direction in the internal space of the cylindrical member 11. The positions at which the acoustic lenses 2 are fitted are set to the positions opposed to the respective ultrasonic vibrators 5. Each of the acoustic lenses 2 has roughly the same shape and roughly the same size in a planar view as those of the ultrasonic vibrator 5, and individually covers the entire surface of opposed one of the ultrasonic vibrators 5.

In the capsule-type housing 10, two sets of ultrasonic transmission/reception units 3 as an element part, and a battery 8 are housed as principal internal constituents. The ultrasonic transmission/reception units 3 are each constituted by a vibrator plate 4, a control board 71, and a flexible wiring board (flexible printed circuits (FPC)) 73. The battery 8 is connected to the control boards 71 of the respective sets via a cable 9, and supplies electrical power necessary for the operation of the ultrasonic probe 1.

The vibrator plate 4 is a plate-like member having the plurality of ultrasonic vibrators 5 arranged, wherein the ultrasonic vibrators are each a transmission/reception section of an ultrasonic wave, and each have a rectangular planar shape. The vibrator plates 4 of the respective sets are each formed to have a semicylindrical shape in an assembly process of the ultrasonic transmission/reception unit 3, and are arranged so that the end surfaces are opposed to each other to form a cylindrical shape as a whole. Further, the vibrator plates 4 are housed in the capsule-type housing 10 so that the respective outer peripheral surfaces are disposed along the inner peripheral surface of the cylindrical member 11, and thus, there is realized a configuration of circularly arranging the plurality of ultrasonic vibrators 5 in the circumferential direction.

It should be noted that the number of ultrasonic transmission/reception units 3 is not limited to two, but can arbitrarily be determined providing the ultrasonic vibrators 5 can circularly be arranged. For example, it is also possible to set the number of the ultrasonic transmission/reception units 3 to be equal to or greater than three, and combine the three or more vibrator plates with each other to form the cylindrical shape as a whole. Alternatively, it is also possible to set the number of the ultrasonic transmission/reception units 3 to one, and shape the single vibrator plate into a cylindrical shape. Further, the ultrasonic vibrator 5 is not limited to the configuration of having the rectangular planar shape, but there can be adopted a configuration of arranging a plurality of ultrasonic vibrators 5 in a line or two or more lines in the rectangular area.

On the control board 71, there are mounted necessary electronic components such as a storage medium such as an IC (integrated circuit) memory, and a transmission/reception circuit for transmitting the received signal of the reflected wave as the measurement result and so onto the receiver located outside the body in addition to a CPU (central processing unit), an ASIC (application specific integrated circuit), an FPGA (field programmable gate array), and a variety of types of integrated circuits. By the CPU and so on executing a program stored in the storage medium in the control board 71, there are realized a variety of functions of the ultrasonic probe 1 such as an ultrasonic measurement function and a function of wirelessly transmitting the measurement result (e.g., the received signal of the reflected wave).

The flexible wiring board 73 electrically connects the vibrator plate 4 and the control board 71 to each other. Through the flexible wiring board 73, the control board 71 outputs a drive signal to each of the ultrasonic vibrators 5 to emit the ultrasonic signal, and at the same time, the received signal of the reflected wave received by each of the ultrasonic vibrators 5 is transmitted to the control substrate 71.

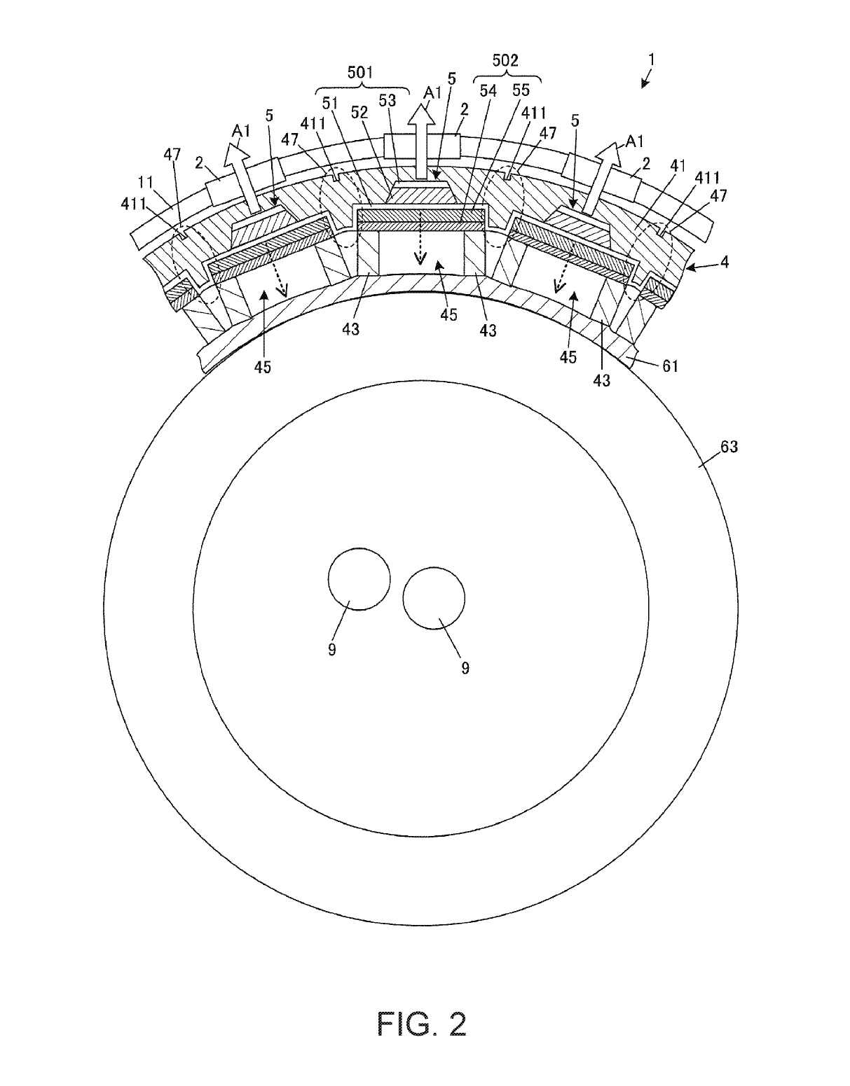

FIG. 2 is a diagram schematically showing a cross-sectional surface of the ultrasonic probe 1 including the vibrator plate 4 viewed along the arrowed line A-A shown in FIG. 1. In FIG. 2, the hatching indicating the cross-sectional surface is partially omitted for the sake of convenience.

As shown in FIG. 2, the vibrator plate 4 has a membrane structure, in which an acoustic matching layer 41 for covering an upper surface side of the ultrasonic vibrators 5 arranged in the circumferential direction, and support members 43 extending below the ultrasonic vibrators 5 to support the ultrasonic vibrators 5 and the acoustic matching layer 41 are provided, and the ultrasonic vibrators 5 are each disposed on a hollow part (cavity) 45 located between the support members 43 adjacent to each other to be paired.

The ultrasonic vibrators 5 are each, for example, a unimorph-type ultrasonic vibrator having an elastic plate 502 disposed on one surface (a lower surface in FIG. 2) of a piezoelectric body 501. It should be noted that the ultrasonic vibrator 5 is not limited to the unimorph-type, but a bimorph-type ultrasonic vibrator can also be used. The piezoelectric body 501 is formed of a lower electrode (also referred to as a lower electrode layer) 51, a thin-film piezoelectric (piezo) element 52, and an upper electrode 53 stacked on one another. The lower electrode 51 is formed as an electrode layer (the lower electrode layer) common to the ultrasonic vibrators 5, and is partially exposed on the lower surface of the vibrator plate 4 between the ultrasonic vibrators 5 adjacent to each other. On the other hand, the elastic plate 502 is formed by stacking a metal thin film (a vibrating plate) 55 such as a zirconia thin film on an oxide film 54 such as a silicon thermal oxide film.

The acoustic matching layer 41 assumes the role of matching the acoustic impedance between the ultrasonic vibrators 5 and a region in the body cavity and converging an ultrasonic beam together with the acoustic lenses 2 provided to the cylindrical member 11 and disposed so as to be opposed to the respective ultrasonic vibrators 5.

It should be noted that although FIG. 2 partially shows the vibrator plate 4, the two vibrator plates 4 are each shaped into the semicylindrical shape and are combined with each other to form the cylindrical shape as described above, and the plurality of ultrasonic vibrators 5 is circularly arranged in the entire circumference along the inner circumferential surface of the cylindrical member 11. Therefore, the ultrasonic probe 1 can radially emit an ultrasonic wave by the individual ultrasonic vibrator 5 transmitting the ultrasonic wave outward in the ultrasonic radiation direction A1 (the direction of the white arrow shown in FIG. 2). According to this configuration, when the ultrasonic probe 1 is introduced in the body cavity to perform the ultrasonic measurement, the ultrasonic measurement can be performed in all directions on the periphery of the capsule-type housing 10 viewed from the longitudinal direction of the capsule-type housing 10 (around the axis along the longitudinal direction).

Here, the acoustic matching layer 41 is a resin layer formed of a resin material having flexibility, and has a configuration of covering the upside of the ultrasonic vibrators 5 and at the same time having the resin layer intervene between the ultrasonic vibrators 5 adjacent to each other. Specifically, the acoustic layer 41 has a function of providing flexibility to the vibrator plate 4 in addition to the original function exerted together with the acoustic lenses 2, and forms flexion sections 47 easy to bend together with the parts of the lower electrode layer 51 exposed on the lower surface of the vibrator plate 4 between the ultrasonic vibrators 5. Therefore, when applying external force for deflecting upward the vibrator plate 4 to the vibrator plate 4, the vibrator plate 4 bends mainly at the flexion sections 47. Further, since the area of the acoustic matching layer 41 has a high proportion of the entire area of the vibrator plate 4, the whole of the vibrator plate 4 also deflects gently.

Further, focusing attention on the lower electrode layer 51, since the electrode (the lower electrode) between the ultrasonic vibrators 5 adjacent to each other can be formed of the lower electrode layer 51, the lower electrode layer 51 makes a contribution to narrowing the arrangement interval between the ultrasonic vibrators 5 adjacent to each other. Further, since the ultrasonic vibrators 5 adjacent to each other are connected to each other, it is possible to keep the binding force for connecting the ultrasonic vibrators 5 adjacent to each other, and since only small thickness of the lower electrode layer 51 is sufficient, there is no chance to hinder the flexibility. Therefore, it can be said that it makes a contribution to the high-density packaging of the ultrasonic vibrators 5.

Further, in the present embodiment, the acoustic matching layer 41 has groove sections 411 on the upper surface side at the positions where the flexion sections 47 are formed (the upper surface positions of the acoustic matching layer 41 located above the areas between the ultrasonic vibrators 5 adjacent to each other). The groove sections 411 are formed in parallel to the longitudinal direction of the ultrasonic vibrators 5 in the areas between the ultrasonic vibrators 5 adjacent to each other when viewed from above (see FIG. 7). According to the groove sections 411, the flexibility of the flexion sections 47 can be improved. As described above, according to the vibrator plate 4, it is possible to arrange the ultrasonic vibrators 5 in the circumferential direction while densely packaging the plurality of ultrasonic vibrators 5.

In the actual assembly process of the ultrasonic transmission/reception unit 3, a fixation member 63 having a cylindrical shape is disposed on the inner side of each of the vibrator plates 4, and the lower surface of each of the support members 43 is bonded to the outer peripheral surface of the fixation member 63 using a sheet-like adhesive member (an adhesive film) 61. Thus, the vibrator plates 4 are each fixed while keeping the semicylindrical shape. Through the inside of the fixation member 63, there are inserted cables 9 for connecting the control board 71 and the battery 8 to each other.

Then, a procedure of assembling the ultrasonic transmission/reception units 3 to manufacture the ultrasonic probe 1 will be described. The ultrasonic transmission/reception units 3 are each assembled by connecting the vibrator plate 4 and the control board 71 to each other via the flexible wiring board 73, and then shaping the vibrator plate 4 into the cylindrical shape. In advance of the assembly, the internal constituents such as the vibrator plates 4 are prepared.

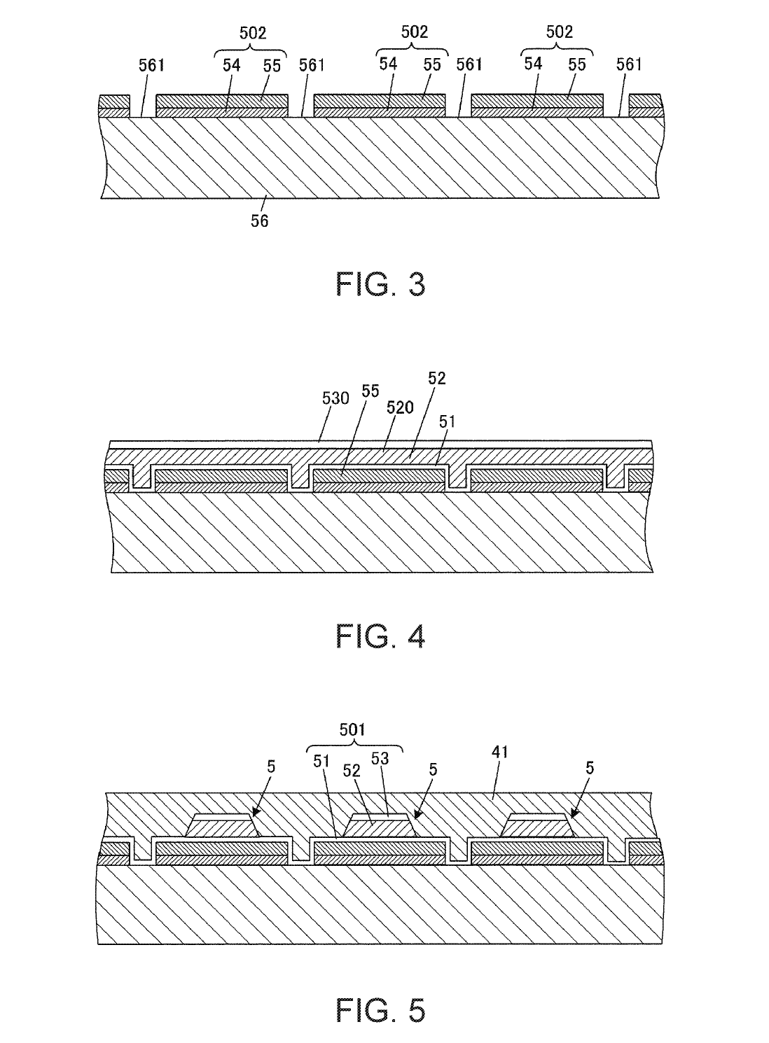

FIG. 3 through FIG. 6 are cross-sectional views showing the manufacturing process of the vibrator plate 4 in a phased manner. Firstly, a silicon thermal oxide film (SiO₂) is formed on a principal surface (the obverse surface) of a silicon substrate 56, and then, a zirconia thin film (ZrO₂) is formed on the upper surface of the silicon thermal oxide film. Then, as shown in FIG. 3, openings 561 reaching the upper surface of the silicon substrate 56 are formed by etching. Thus, there can be obtained the elastic plates 502 of the respective ultrasonic vibrators 5, each having the metal thin film 55 formed of the zirconia thin film (ZrO₂) disposed on the oxide film 54 formed of the silicon thermal oxide film (SiO₂), at predetermined intervals to be the flexion sections 47 in a posterior process.

Subsequently, as shown in FIG. 4, an electrode film is deposited on the upper surface of the metal thin film 55 to form the lower electrode layer 51. On this occasion, necessary interconnections (interconnections for the lower electrodes) for connecting the lower electrode layer 51 to wiring patterns of the flexible wiring board 73 are formed in advance. Then, a thin-film piezoelectric element layer 520 to be the thin-film piezoelectric (piezo) elements is formed on the upper surface of the lower electrode layer 51, and then an electrode film is deposited on the upper surface of the thin-film piezoelectric element layer 520 to form an upper electrode film 530.

Then, as shown in FIG. 5, an opening reaching the upper surface of the lower electrode layer 51 is formed by etching in each of the interelement areas to complete element separation. The remaining parts of the thin-film piezoelectric element layer 520 (see FIG. 4) turn to the thin-film piezoelectric elements 52, and the remaining parts of the upper electrode film 530 (see FIG. 4) turn to the upper electrodes 53. Further, on this occasion, interconnections for the upper electrodes for connecting each of the upper electrodes 53 to the wiring patterns of the flexible wiring board 73 are formed in advance. The interconnections for the upper electrodes are formed in one end part (connection side end part) in the short axis direction of the vibrator plate 4, to which the flexible wiring board 73 is connected in the assembly process of the ultrasonic transmission/reception unit 3, so as not to overlap a blank space 481 described later (see FIG. 7). The same applies to the interconnections for the lower electrodes described above. Thus, there can be obtained the ultrasonic vibrators 5 each having the piezoelectric body 501, which has the lower electrode 51, the thin-film piezoelectric element 52, and the upper electrode 53 stacked on one another, disposed on the elastic plate 502. Subsequently, the entire area on the upper surface side of the ultrasonic vibrators 5 is filled with the resin material to form the acoustic matching layer 41.

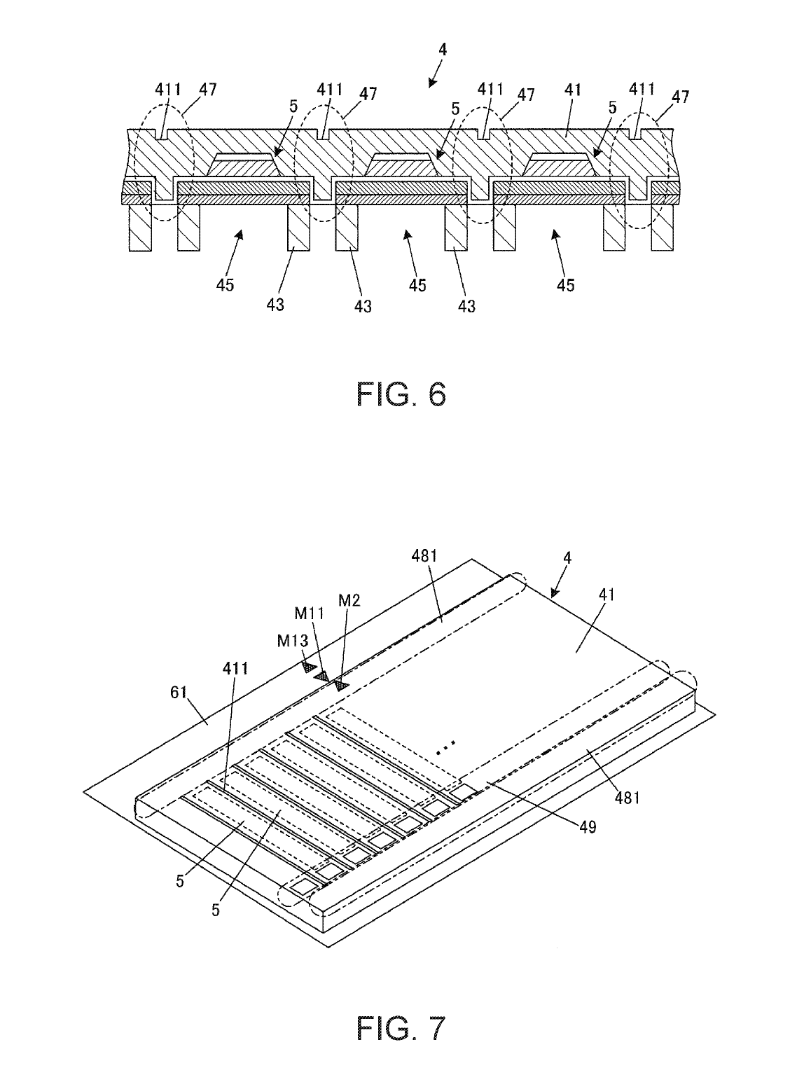

Subsequently, as shown in FIG. 6, a grinding process is performed on the reverse surface of the silicon substrate to expose the elastic plate 502 below the ultrasonic vibrators 5 to thereby form cavities 45, and at the same time expose the lower electrode layer 51 located between the ultrasonic vibrators 5 adjacent to each other to thereby form the support members 43, and thus the flexion sections 47 are obtained. Further, at the upper surface positions of the acoustic matching layer 41 to be the flexion sections 47, there are formed the groove sections 411.

An assembly procedure of the ultrasonic transmission/reception unit 3 will be described below. FIG. 7 through FIG. 10 are perspective views showing the assembly procedure in a phased manner. It should be noted that the configuration of the ultrasonic vibrators 5 and so on is shown schematically.

Firstly, as shown in FIG. 7, the adhesive film 61 is pasted on the lower surface of the vibrator plate 4 (bonding process). The adhesive film 61 is provided with a marker for alignment in advance using an appropriate method. In more detail, the adhesive film 61 is provided with a first marker M11 for alignment with the vibrator plate 4, and a second marker M13 for alignment with the fixation member 63 to be used in a shaping process in a posterior stage. On the other hand, the upper surface of the vibrator plate 4 is also provided with a vibrator plate side marker M2 for alignment with the adhesive film 61 in advance at an appropriate position. In the bonding process, the adhesive film 61 is disposed on the lower surface side of the vibrator plate 4 so that a pointing tip of the first marker M11 corresponds to a pointing tip of the vibrator plate side marker M2, and then the bottom surface of each of the support members 43 is pasted to the obverse surface of the adhesive film 61.

Here, the acoustic matching layer 41 is disposed on the upper surface side of the vibrator plate 4, and the groove sections 411 are formed on the upper surface of the acoustic matching layer 41 as described above. However, the groove sections 411 are formed remaining the blank spaces 481 with a predetermined width on both ends in the short axis direction of the vibrator plate 4. This is because if the grooves are formed up to the end surface, it becomes unachievable to keep the plane state, and the workability in the bonding process and a connection process in the posterior stage becomes worse. Further, the interconnections for the upper electrodes and the interconnections for the lower electrodes described above are formed in an interconnection forming area 49 located on the inner side of the blank space 481 in the connection side end part. It should be noted that it is also possible to arrange that the groove sections 411 are formed up to the end surface by assuring the workability with another measure such as a rod-like member as a temporary splint detachably attached to each of the ends in the short axis direction (the long sides in the top view in FIG. 7) of the vibrator plate 4.

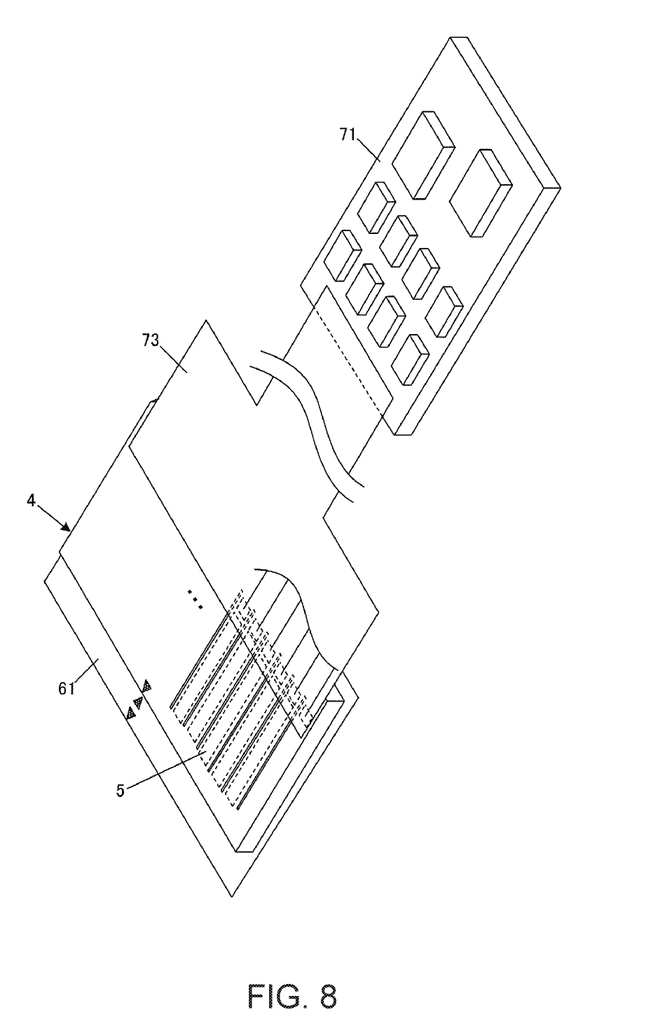

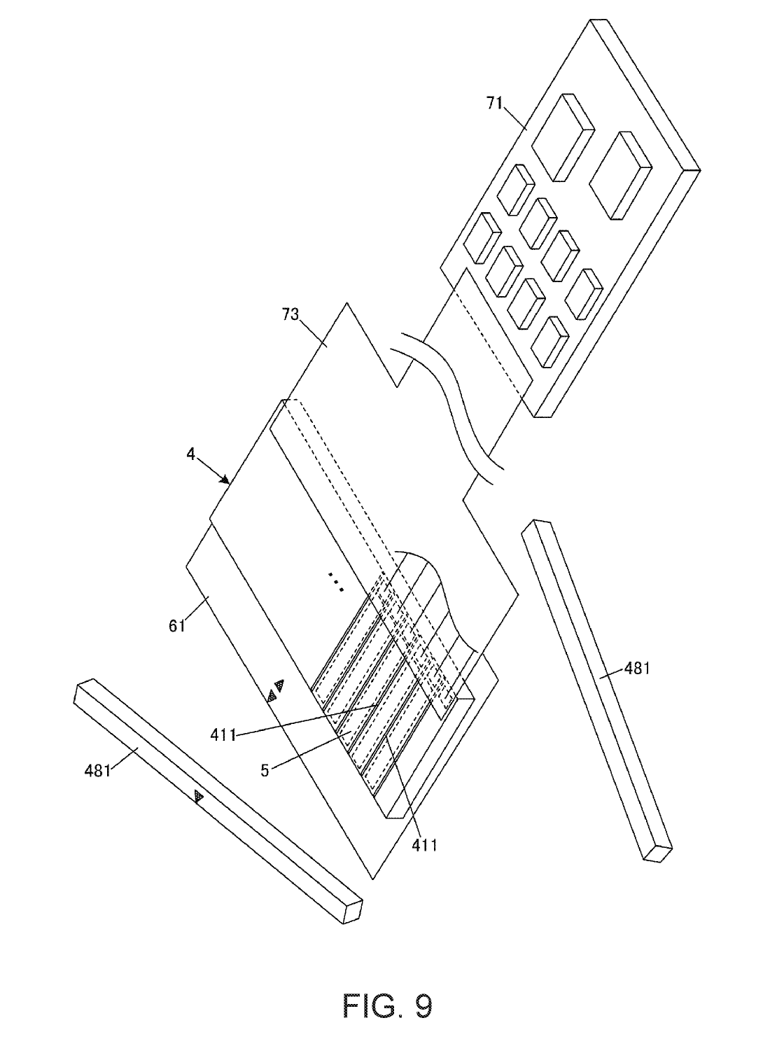

In the connection process as a subsequent process, as shown in FIG. 8, the vibrator plate 4 is connected to one end side of the flexible wiring board 73. On this occasion, the interconnections for the lower electrodes and the interconnections for the upper electrodes in the interconnection forming area 49 are connected to the wiring patterns of the flexible wiring board 73. It should be noted that to the other end side of the flexible wiring board 73, there is connected the control board 71. Due to the connection process, the vibrator plate 4 and the control board 71 are electrically connected to each other via the flexible wiring board 73.

After finishing the connection process, as shown in FIG. 9, the blank spaces 481 are removed by cutting with processing equipment such as a laser scribing apparatus.

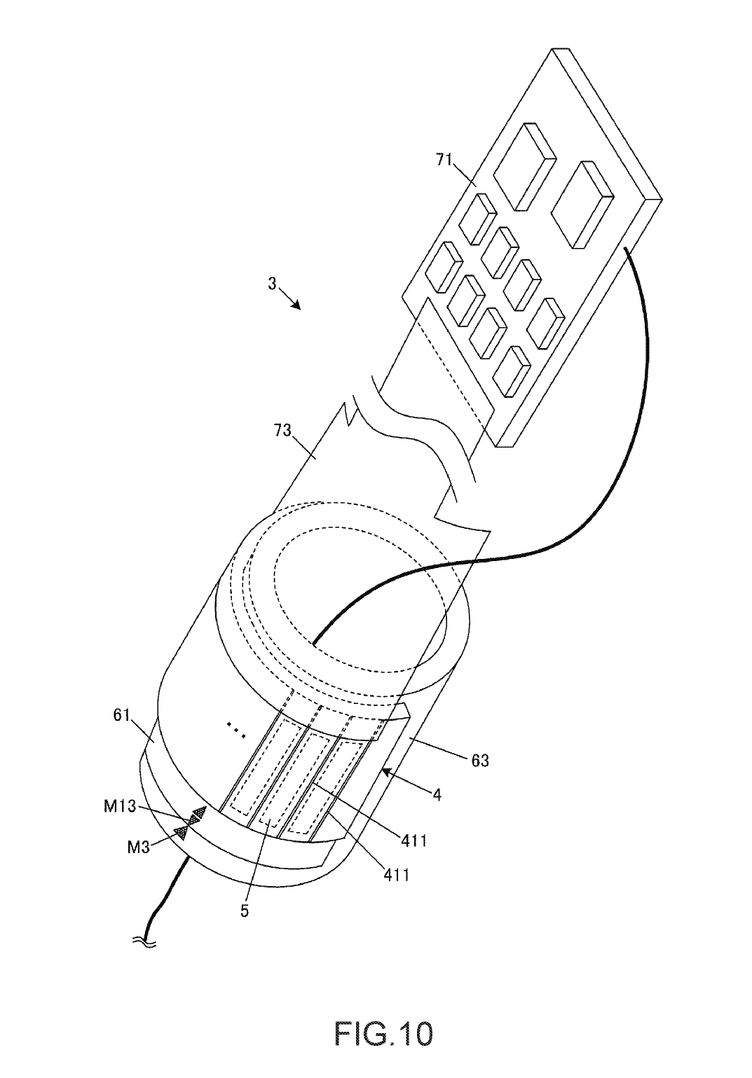

Subsequently, the process proceeds to the shaping process shown in FIG. 10. In the shaping process, the reverse surface of the adhesive film 61 is pasted to the outer peripheral surface of the fixation member 63 to thereby bond the vibrator plate 4 to the fixation member 63. The fixation member 63 is provided with the marker M3 in advance at the appropriate position. It should be noted that since the vibrator plate 4 constituting another set of ultrasonic transmission/reception unit 3 is bonded to the fixation member 63 in substantially the same manner, the fixation member side marker is provided to the opposite position at an interval of 180° with the fixation member side marker M3 in the same manner.

Further, the fixation member 63 is disposed below the adhesive film 61 so that a pointing tip of the fixation member side marker M3 corresponds to a pointing tip of the second marker M13, and then the outer peripheral surface of the fixation member 63 is pasted to the reverse surface of the adhesive film 61. On this occasion, the flexible wiring board 73 appropriately deforms between the vibrator plate 4 and the control board 71 to keep the connection state with the vibrator plate 4 and the control board 71. Thus, the vibrator plate 4 is shaped into the semicylindrical shape, and is fixed.

Hereinabove, the assembly of a set of ultrasonic transmission/reception unit 3 is completed. The remaining set of ultrasonic transmission/reception unit 3 is also assembled in substantially the same manner, and the shaping process in that case is performed by bonding another vibrator plate 4 to the fixation member 63 to which the vibrator plate 4 has been bonded in advance. The operation is performed using the other fixation member side marker as a mark. Subsequently, the cables 9 for connecting the control boards 71 of the respective sets to the battery 8 are inserted through the inside of the fixation member 63, and then the ultrasonic transmission/reception units 3 are housed in the capsule-type housing 10 together with other internal constituents.

As described hereinabove, according to the present embodiment, it is possible to densely package the ultrasonic vibrators 5 to circularly arrange the ultrasonic vibrators 5 along the circumferential direction, and to realize the ultrasonic probe 1 achieving further miniaturization. Due to the further miniaturization, the burden on the test subject when swallowing the ultrasonic probe 1 can be reduced compared to one in the related art. Further, since the high-density packaging of the ultrasonic vibrators 5 becomes possible, it becomes possible to obtain a high-definition ultrasonic image.

It should be noted that the ultrasonic vibrator 5 having the configuration explained in the embodiment described above transmits the ultrasonic wave also in the direction of the arrowed dotted line opposite to the arrow A1 in FIG. 2. Therefore, it is also possible to adopt a configuration inverse to that in FIG. 2, that is a configuration in which the elastic plate 502 of the ultrasonic vibrator 5 is disposed on the upper side, and the piezoelectric body 501 is disposed on the lower side.

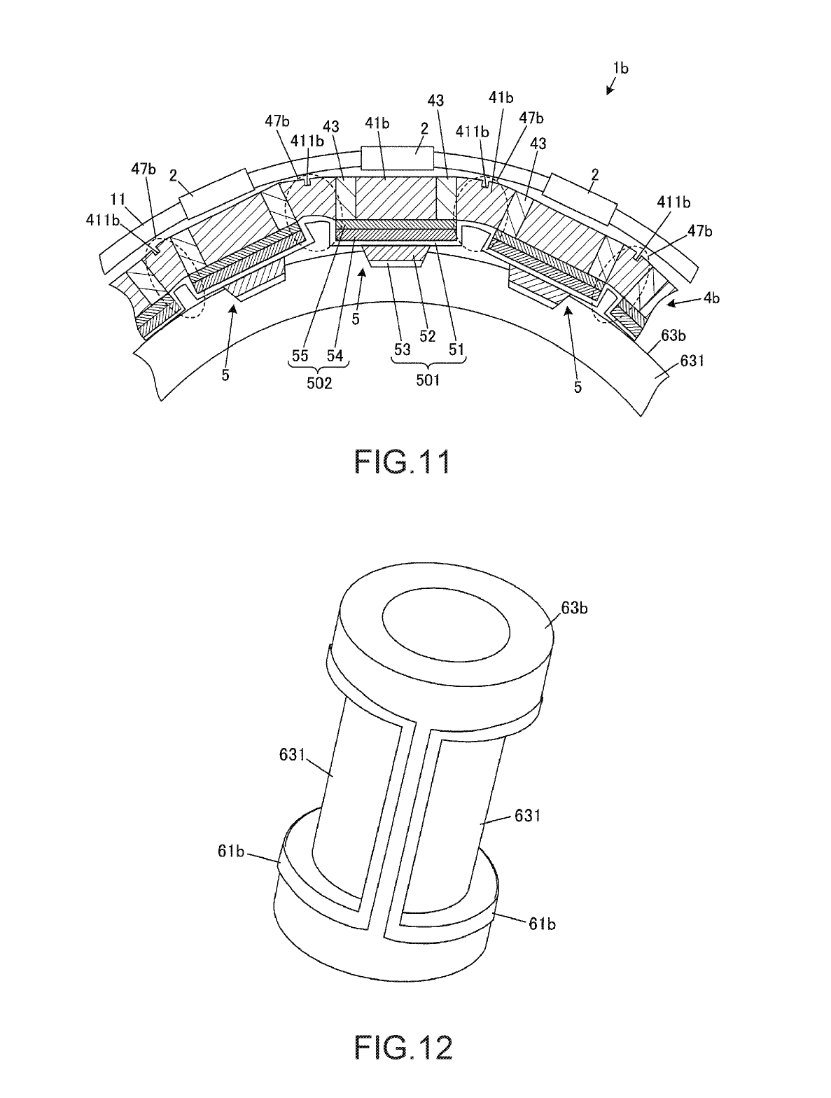

FIG. 11 is a cross-sectional view of an ultrasonic probe 1b including a vibrator plate 4b according to the present modified example. In FIG. 11, substantially the same constituents as in the embodiment described above are denoted by the same symbols. As shown in FIG. 11, in the vibrator plate 4b according to the present modified example, a space between the support members 43 adjacent to each other is filled with a resin material. Thus, an acoustic matching layer 41b made of a resin material is disposed above the elastic plates 502 constituting the respective ultrasonic vibrators 5 facing upward, and at the same time, flexion sections 47b between the ultrasonic vibrators 5 adjacent to each other. The flexion sections 47b each include a part of the lower electrode layer 51 exposed between the ultrasonic vibrators 5 adjacent to each other similarly to the embodiment described above. On the upper surface, there are formed groove sections 411b.

FIG. 12 is a perspective view showing a fixation member 63b of the present modified example. In the present modified example, the side surface of the fixation member 63b for bonding to fix the vibrator plate 4b is provided with recessed sections 631. This is for preventing the piezoelectric body 501 of each of the ultrasonic vibrators 5 facing upward from having contact with the fixation member 63b. In each of the recessed sections 631, there is positioned an area (a vibrator area), which is located on the lower surface side of the vibrator plate 4b, and in which the ultrasonic vibrators 5 are arranged.

The fixation member 63b according to the present modified example is prepared assuming the case of combining the two vibrator plates 4b to each other to form the cylindrical shape similarly to the embodiment described above, and the side surface of the fixation member 63b is provided with the two recessed sections 631 formed respectively for the two vibrator plates 4b. Further, when assembling the ultrasonic transmission/reception unit, an adhesive film 61b shaped like a frame is disposed so as to surround the opening of the recessed section 631, and the outer circumferential edge part of the vibrator plate 4b is bonded and fixed to the fixation member 63b.

Further, although in the embodiment described above, the ultrasonic probe 1 introduced in the body cavity is described, the measurement object is not particularly limited, and is not required to be a living body.

Further, besides the capsule type, the invention can also be applied to an ultrasonic probe for performing the ultrasonic measurement while having contact with a surface of the body. Specifically, the vibrator plate 4 explained in the embodiment described above can be bent at the flexion sections 47 located between the ultrasonic vibrators 5 adjacent to each other. Therefore, if a contact-type ultrasonic probe is configured using the vibrator plate 4, the ultrasonic probe can closely be attached to the body surface of a desired measurement region such as an arm, a finger, or an abdomen when performing the ultrasonic measurement, and is therefore convenient.

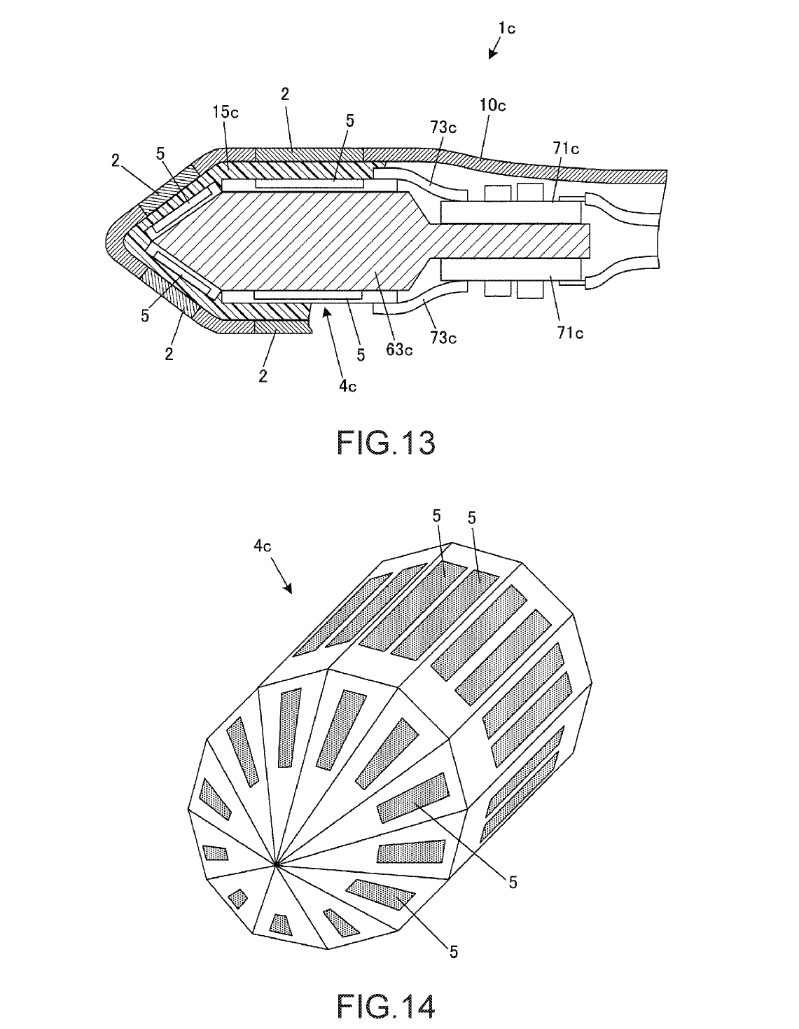

Further, from a viewpoint of miniaturization, the invention can also be applied to a catheter-type ultrasonic probe such as one for intravascular ultrasonography or one for an endoscope. FIG. 13 is a schematic diagram showing an internal configuration example of the catheter-type ultrasonic probe 1c according to the present modified example, and shows the internal condition with some constituents shown in a cross-sectional view.

As shown in FIG. 13, the ultrasonic probe 1c according to the present modified example is provided with a vibrator plate 4c having a plurality of ultrasonic vibrators 5 arranged, a control board 71c, and a flexible wiring board 73c housed in an exterior case 10c, and is mounted on an inner tip of an insertion tube having flexibility. The acoustic lenses 2 are fitted into the exterior case 10c at the positions opposed to the respective ultrasonic vibrators 5. Further, the inside is filled with a resin material 15c, and thus, the acoustic layer is disposed on each of the ultrasonic vibrators 5. The flexible wiring board 73c electrically connects the vibrator plate 4c and the control board 71c to each other.

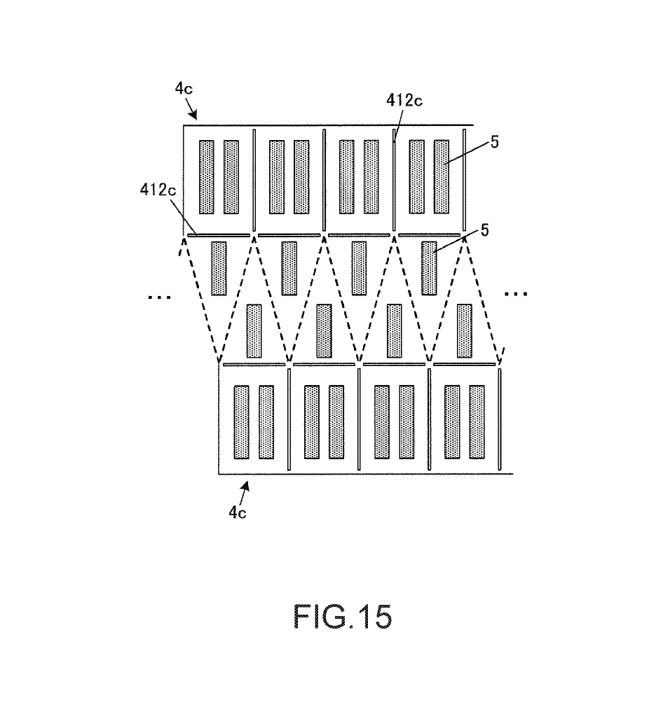

FIG. 14 is a perspective view schematically showing the vibrator plate 4c, and FIG. 15 is a plan view showing the developed state of the vibrator plate 4c before being assembled to be in the state shown in FIG. 14. As shown in FIG. 14, the vibrator plate 4c has an outer shape with the body part shaped like a polygonal column and a tip part shaped like a polygonal pyramid, and the ultrasonic vibrators 5 are disposed on the respective faces except the back end face. It should be noted that the number of the ultrasonic vibrators 5 disposed on the respective faces is not particularly limited, but it is possible to dispose the ultrasonic vibrators 5 on all of the faces one-by-one, or to arrange a plurality of ultrasonic vibrators 5 on all of the faces.

According to the ultrasonic probe 1c, the arrangement of the ultrasonic vibrators 5 in the circumferential direction can be realized by the body part shaped like a polygonal column having the ultrasonic vibrators 5 disposed on the respective faces. Further, in the present modified example, by disposing the ultrasonic vibrators 5 also on the tip part, it is possible to transmit and receive the ultrasonic wave frontward in the insertion direction of the insertion tube when used.

As shown in, for example, FIG. 15, the vibrator plate 4c can be obtained by cutting a single plate along the dotted lines. Specifically, the ultrasonic vibrators 5 are formed on the silicon substrate in substantially the same manner as in the embodiment described above, and then the silicon substrate is cut. On this occasion, by forming slits 412c at positions where sides are formed when assembled to make the vibrator plate 4c easy to bend, and then bending the vibrator plate 4c along the slits 412c, the vibrator plate 4c can be assembled. In the actual assembly process, a fixation member 63c shown in FIG. 13, the tip side of which has the same shape as the outer shape of the vibrator plate 4c, is disposed inside the vibrator plate 4c, and then the faces of the vibrator plate 4c forming the inner side are bonded and fixed to the side surfaces of the fixation member 63c.

Further, in the case of adopting the polygonal column and the polygonal pyramid as the shapes of the body part and the tip part of the vibrator plate 4c as in the present modified example, by making the width of the plate to be cut equal to the width obtained by adding the height of a triangle forming the side face of the tip part to a length twice as large as the length of the body part as shown in FIG. 15, the two vibrator plates 4c can be obtained without waste. It should be noted that the shapes of the body part and the tip part are not limited to those described above, but a cylindrical shape and a conical shape can also be adopted, and it is also possible to adopt a configuration in which the tip part is shaped like a truncated pyramid, a circular truncated cone, or the like, and the ultrasonic vibrator is also disposed on the flat face.

The entire disclosure of Japanese Patent Application No. 2016-054950 filed on Mar. 18, 2016 is expressly incorporated by reference herein.

What is claimed is:

1. An ultrasonic probe comprising:
a plurality of ultrasonic vibrators arranged in a circumferential direction;
an acoustic matching layer disposed on the ultrasonic vibrators; and
a flexion section disposed between the ultrasonic vibrators adjacent to each other in the circumferential direction, and having an electrode layer acting as an electrode of the ultrasonic vibrators adjacent to each other.

2. The ultrasonic probe according to claim 1, wherein
the electrode layer is exposed in the flexion section.

3. The ultrasonic probe according to claim 1, wherein
the acoustic matching layer is disposed on the ultrasonic vibrators and between the ultrasonic vibrators, and formed of a material having flexibility, and
a principal element in a thickness direction of the flexion section is constituted by the acoustic matching layer and the electrode layer.

4. The ultrasonic probe according to claim 1, wherein
the flexion section has the electrode layer on one end side in a thickness direction, and has a groove section adapted to improve flexibility on an end surface on the other end side.

5. The ultrasonic probe according to claim 1, wherein
the ultrasonic vibrators are circularly arranged with an ultrasonic radiation direction facing outward, and
an ultrasonic wave can be transmitted and received throughout an entire circumference.

6. The ultrasonic probe according to claim 5, wherein
N (N≧2) element parts each including the ultrasonic vibrators, the acoustic matching layer, and the flexion part are arranged in the circumferential direction to configure the circular arrangement of the ultrasonic vibrators.

7. The ultrasonic probe according to claim 1, further comprising:
a shell part incorporating all of the constituents to introduce the ultrasonic probe in a body cavity.

8. The ultrasonic probe according to claim 7, wherein
the shell part has an elongated shape,
the ultrasonic vibrators are provided to a body part of the shell part, and
an ultrasonic wave is radially emitted viewed from a longitudinal direction of the shell part.

9. The ultrasonic probe according to claim 7, wherein
the shell part has a capsule shape.

10. The ultrasonic probe according to claim 1, further comprising:
a transmitter adapted to transmit information base on an echo measured by the ultrasonic vibrators.

11. An ultrasonic apparatus comprising:
the ultrasonic probe according to claim 1; and
an image generation device having a receiver adapted to receive the information transmitted by the transmitter, and adapted to generate an image based on the information.
